(19) United States
(12) Patent Application Publication
(10) Pub. No.: US 2009/0104297 A1
(43) Pub. Date: Apr. 23, 2009

(54) HERBAL COMPOSITION FOR TREATMENT OF INFECTIONS CAUSED BY DERMATOPHYTES

(75) Inventors: Kavita Kadam, Maharashtra (IN); Muthusamy Veluchamy Shanmuganathan, Tamil Nadu (IN); Dhananjay Sapre, Maharashtra (IN)

Correspondence Address: MERCHANT & GOULD PC, P.O. BOX 2903, MINNEAPOLIS, MN, 55402-0903, US

(73) Assignee: Piramal Life Sciences Limited, Mumbai (IN)

(21) Appl. No.: 12/293,300

(22) PCT Filed: Mar. 27, 2007
(86) PCT No.: PCT/IB2007/051062

§ 371 Date: Sep. 17, 2008

(30) Foreign Application Priority Data
Mar. 29, 2006 (IN) 478/MUM/2006

(51) Int. Cl. A61K 36/75; A61K 31/403; A61P 31/10

(52) U.S. Cl. 424/769; 514/411

(57) ABSTRACT

The present invention relates to a novel herbal composition comprising extract of roots of the plant Murraya koenigii for the treatment of infections caused by dermatophytes, particularly tinea infections, and method of manufacture of said composition. The said herbal composition comprises 2-methoxy-3-methyl-9H-carbazole (compound 1) as a bioactive ingredient. Invention also discloses methods for topical application of the said herbal composition for the treatment of infections caused by dermatophytes.

### FIELD OF INVENTION

The present invention relates to a novel herbal composition comprising plant extract as an active ingredient and a method of manufacture of the same. The invention relates to the method of treatment of various dermatophyte infections by adapting the said composition in mammals. More particularly, the present invention relates to an anti-dermatophyte herbal composition, effective against tinea infections, utilizing an extract obtained from roots of the plant, Murraya koenigii.

### BACKGROUND OF INVENTION

During the last decade there has been an important increase in the incidence of fungal infections. Fungal infections occur worldwide and affect all ages and all races. However, prevalence of the organisms varies by country. Millions of people throughout the world are affected by superficial fungal infections, which are the most common skin diseases (Postgrad. Med. 91, 239-244 and 249-252, (1992); Drugs 52, 209-224, (1996)). These infections, which occur in both healthy and immunocompromised persons, are caused by dermatophytes, yeasts and nondermatophyte molds. Approximately ninety percent of fungal skin infections are caused by dermatophytes. Dermatophytes are fungal organisms which infect keratin of humans and animals, affecting the skin, nails and hair. Dermatophytes are classified as anthropophilic, zoophilic or geophilic according to their normal habitat. Anthropophilic dermatophytes are restricted to human hosts and produce a mild, chronic inflammation. Zoophilic organisms are found primarily in animals and infect a human who have contact with infected animals such as cats, dogs, cattle, horses, birds, or other animals. Geophilic species are usually recovered from the soil but occasionally infect humans and animals. They cause a marked inflammatory reaction, which limits the spread of the infection and may lead to a spontaneous cure but may also leave scars. There are three genera of dermatophytes, called Trichophyton (affects skin hair and nails) Microsporum (a type of fungus that causes ringworm epidemics in children) and Epidermophyton (a type of fungus which grows on the outer layer of the skin and is the cause of tinea). Commonly the fungal skin infections caused by dermatophytes are the tinea infections. Tinea infections are contagious and can be passed through direct contact or by contact with clothing, from shower and pool surfaces, and even from pets. The estimated lifetime risk of acquiring a dermatophyte infection is between ten and twenty percent (J. Am. Acad. Dermatol. 34, 282-286, (1996)).

Recognition and appropriate treatment of these infections reduces both morbidity and discomfort and lessens the possibility of transmission. The present line of treatment involves use of anti-fungals, such as tolnaftate, terbinafine hydrochloride, griseofulvin and imidazoles such as ketoconazole, miconazole nitrate and clotrimazole. Griseofulvin is used for systemic therapy. Generally the treatment using anti-fungals requires administering the drug two or three times a day for at least ten to fourteen days, and for some medications it may even extend for up to four weeks. Terbinafine hydrochloride taken in tablet form may have to be taken for considerable lengths of time, potentially for months. It is a common treatment to apply the topical anti-fungal for two weeks after the skin is healed, to eradicate all remaining fungal spores. Reoccurrences of the infection are frequent, and for some patients, such as those who suffer from diabetes or circulatory problems, tinea infections and their treatment can be quite serious.

Herbal medicines include treatment and cure for many ailments through extracts of plant materials. Various combinations have been found effective in treating and curing diseases affecting mankind. Herbs have long been known and used throughout the world for treatment of many conditions, including skin conditions, and there is at least some evidence that herbal remedies may tend to have less deleterious side effects than corresponding synthetic drugs. Even with herbal treatments, however, numerous difficulties are encountered in the treatment of medical conditions. A single herb may contain numerous active, and sometimes conflicting, components. So, it is important to ascertain the effects/side effects of the herbal extract by establishing the presence and characterization of the active ingredient.

Herbs traditionally known or used for treating athlete's foot specifically include tea tree, garlic, goldenseal and various parts of the black walnut tree, which is known to be toxic.

A herbal composition for the treatment of tinea infections is described in the U.S. Pat. No. 6,254,897 (U.S. '897 patent). The U.S. '897 patent particularly describes a composition effective against tinea infections utilizing natural substances obtained from a combination of Angelicae pubescentis Radix, Notopterygium Radix and Haliotis diversicolor Reeve. However, the patent does not contain information regarding the active ingredient present in the plant extract/s, which is responsible for anti-tinea activity. Moreover, the patent does not teach the synergistic effect of the plant extracts used. Since it is a mixture of three or more plant extracts, it is required to collect or cultivate more than one plant.

Hence, there is a need to develop a new composition, which can overcome the above-mentioned problems associated with the synthetic preparations or herbal extracts used at present for the treatment of tinea infections.

Murraya koenigii commonly known as curry leaf tree belongs to the family Rutaceae. In Ayurveda system of medicine it is recommended for vitiated conditions of kapha and pitta.

Murraya koenigii is an aromatic shrub or small tree with slender but strong woody stem and branches covered with dark gray bark closely crowded by dark green leaves, which are very strongly aromatic. The roots are woody with few branched wiry rootlets and with or without woody sideroots, covered with a thick soft bark. The root bark is aromatic, pungent and slightly bitter in taste. (Pharmacognosy of Indigenous Drugs, Vol I, 433-440, 1982, Central Council for Research in Ayurveda and Siddha, New Delhi.)

Carbazoles which were isolated from leaves of the plant Murraya koenigii were found to be active against Gram-positive and Gram-negative bacteria and fungi. (Indian Journal of Chemistry, Volume 40B, 490-494 (2001)).

Since ancient times the stem bark of Murraya koenigii has been used against eruptions and bites of poisonous animals.

A benzoisofuranone derivative and carbazole alkaloids isolated from the stem bark of Murraya koenigii were evaluated for antimicrobial activity. (Phytochemistry, 66 (13), 1601-1606, (2005)).

One study involved testing of antifungal activity of three compounds namely murrayanine, girinimbine, and mahanimbine isolated from the stem bark of Murraya koenigii (Experientia 21(6), 340. (1965)).

The ethanol extract of roots of Murraya koenigii is reported to have anti-inflammatory, antibacterial and insect antifeedant activity. (Journal of Indian Chemical Society, 70 (7), 655-659, (1993)).

Hithertofore there are no reports on the anti-dermatophyte activity tested for root extract of Murraya koenigii. The present inventors have found that the extract obtained from roots of Murraya koenigii exhibited significant anti-dermatophyte activity against a panel of dermatophytes including Trichophyton mentagrophytes and Microsporum gypseum.

Moreover, there is no report of any composition comprising Murraya koenigii extract for dermatophyte treatment. To overcome the problems of side effects of present line of treatment and to reduce the cost of treatment, the present inventors prepared a novel herbal composition having anti-dermatophyte activity effective against tinea infections, utilizing extract obtained from the plant Murraya koenigii, comprising effective amount of 2-methoxy-3-methyl-9H-carbazole as bioactive ingredient, which mainly contributes to the anti-dermatophyte activity exhibited by the extract.

### OBJECTS OF INVENTION

An object of the present invention is directed at providing a novel composition comprising extract of roots of the plant Murraya koenigii with pharmaceutically acceptable carriers.

Yet, another object of present invention is to provide a herbal composition comprising extract of Murraya koenigii, comprising effective amount of bioactive ingredient 2-methoxy-3-methyl-9H-carbazole, for treatment of dermatophyte infections.

A further object is to provide a method of manufacture of the composition comprising extract of roots of Murraya koenigii.

Another object of the present invention is to provide a herbal composition comprising extract of Murraya koenigii for the treatment of dermatophyte infections.

Another object of the present invention is to provide a herbal composition comprising extract of Murraya koenigii for treating tinea infections effectively.

Yet another objective of the present invention is to provide a pharmaceutical composition comprising extract of Murraya koenigii in combination with other bioactive substances in an effective amount to obtain a synergistic effect for the treatment of tinea infection.

Yet another objective of the invention is to provide a pharmaceutical composition comprising extract of Murraya koenigii in combination with at least one other herbal extract in an effective amount to obtain a synergistic effect for the treatment of tinea infection.

Other objects and further scope of applicability of the present invention will become apparent from the detailed description to follow.

### SUMMARY OF INVENTION

Thus according to one aspect of the present invention there is provided a herbal composition comprising extract of roots of the plant Murraya koenigii with pharmaceutically acceptable carriers.

According to another aspect of present invention there is provided a herbal composition comprising extract of Murraya koenigii, comprising effective amount of bioactive ingredient 2-methoxy-3-methyl-9H-carbazole, for treatment of dermatophyte infections.

According to another aspect of the present invention there is provided method of manufacturing herbal compositions comprising extract of roots of Murraya koenigii.

According to further aspect of the present invention there is provided a herbal composition comprising extract of roots of Murraya koenigii useful for treating dermatophyte infections.

According to further aspect of the present invention there is provided a herbal composition comprising extract of roots of Murraya koenigii useful for treating tinea infections.

According to a further aspect of the present invention there is provided a pharmaceutical composition comprising extract of Murraya koenigii in combination with other bioactive substances in an effective amount to obtain a synergistic effect for the treatment of tinea infection.

According to a further aspect of the present invention there is provided a pharmaceutical composition comprising extract of Murraya koenigii in combination with at least one other herbal extract in an effective amount to obtain a synergistic effect for the treatment of tinea infection.

Other objects and further scope of applicability of the present invention will become apparent from the detailed description to follow. It should be understood, however, that the detailed description and specific examples, while indicating embodiments of the invention, are given by way of illustration only, since various changes and modifications within the spirit and scope of the invention will become apparent to those skilled in the art.

### DETAILED DESCRIPTION OF THE INVENTION

Accordingly the present invention relates to a herbal composition comprising extract of the plant Murraya koenigii for the treatment of infections caused by dermatophytes. As described herein before, the dermatophytes are a group of fungi that invade the dead keratin of skin, hair, and nails. Several species of dermatophytes infect humans; these belong to the Epidermophyton, Microsporum, and Trichophyton genera. Dermatophytoses, commonly known as ringworm or tinea, represent superficial fungal infections caused by dermatophytes, which are among the most common infections encountered in medicine. Tinea infections include tinea capitis which is fungal infection of the scalp that can cause hair loss; tinea barbae which is fungal infection in the beard; tinea corporis which is fungal infection of the skin other than beard, scalp, groin, hands or feet; tinea cruris which is fungal infection of the groin and perineum; tinea pedis which is fungal infection of the feet, known as athlete's foot; tinea manuum which is fungal infection of the hands and tinea unguium which is fungal infection of the nails (Postgrad. Med. 91, 239-244 and 249-252, (1992)).

Thus, the present invention provides a novel herbal composition comprising an extract of Murraya koenigii for treating infections caused by dermatophytes, particularly tinea infections which are the most common skin fungal infections.

In an embodiment, the invention provides a pharmaceutical composition comprising standardized extract of Murraya koenigii along with other pharmaceutical carriers. “Murraya koenigii extract” mentioned here means a blend of compounds present in the plant Murraya koenigii. Such compounds may be extracted from the dried roots of the plant using extraction procedures well known in the art (e.g., the use of organic solvents such as lower alcohols, alkyl esters, alkyl ethers, alkyl ketones, chloroform, petroleum ether, hexane and/or inorganic solvents such as water). The present process for extraction of phytoconstituent derivatives from roots of Murraya koenigii can be scaled up for large scale preparation.

Murraya koenigii extract can be standardized using conventional techniques such as HPLC or HPTLC.

The preliminary activity determination of the extracts may be carried out using various well-known biological assays. Bioactive ingredients may be identified using various techniques such as fractionation on preparative TLC or HPLC.

Bioactive ingredients may be isolated from the extract of roots of Murraya koenigii by bioactivity guided column chromatographic purification and preparative high performance liquid chromatography (HPLC). Compounds may be characterized by analysis of the spectral data.

In one embodiment, the Murraya koenigii extract contains a compound, 2-methoxy-3-methyl-9H-carbazole (Compound 1) as one of the bioactive ingredients. The extract contains 6-10% of 2-methoxy-3-methyl-9H-carbazole, which can be estimated using conventional assay techniques such as high performance thin layer chromatography (HPTLC) or high performance liquid chromatography (HPLC).

The invention is further directed to a method of manufacturing compositions useful for treating dermatophyte infections. The standardized extract of Murraya koenigii is mixed with pharmaceutically acceptable carriers and formulated into therapeutic dosage forms.

The extract of roots of Murraya koenigii is used to prepare topical preparations containing 2.0-20% by weight of the said extract which is thoroughly blended into a conventional base as will be hereafter described in detail. The said herbal composition contains approximately 0.1-2.0% (w/w) of 2-methoxy-3-methyl-9H-carbazole, (compound 1) as bioactive ingredient which is sufficient to achieve the desired results.

The extract of roots of Murraya koenigii is used to prepare topical preparations containing 2.0-20% by weight of the root extract, preferably from about 2.5-10% (w/w), which is thoroughly blended into a conventional base as will be hereafter described in detail. It is to be noted that for most of the conditions targeted, the said herbal composition having preferably 0.15-1.0% (w/w) of 2-methoxy-3-methyl-9H-carbazole as bioactive ingredient, is sufficient to achieve the desired results.

By “pharmaceutically acceptable” it is meant the carrier, diluent, excipients, and/or salt must be compatible with the other ingredients of the formulation, and not deleterious to the recipient thereof.

As used herein, “topical application” or topical administration” means directly laying on or spreading on outer skin using, e.g., by use of the hands or an applicator such as a wipe.

The topical compositions useful in the present invention involve formulations suitable for topical application to skin. The compositions may be formulated into a wide variety of product types that include but are not limited to lotions, creams, gels, dusting powders, wax based sticks, sprays, ointments, cleansing liquid washes and solid bars, shampoos, pastes, mousses, wipes, patches, wound dressing and adhesive bandages, hydrogels, films and cosmetics. The formulation may be applied two or three times a day to achieve the desired result.

In an embodiment of this invention, the topical compositions of the present invention can be formulated into a dusting powder.

In another embodiment of this invention, the topical compositions of the present invention can be formulated into a cream.

As used herein, the term “pharmaceutically acceptable carrier” means a non-toxic, inert solid, semi-solid, diluent, encapsulating material or formulation auxiliary of any type. Some examples of materials which can serve as pharmaceutically acceptable carriers are: starches such as corn starch and potato starch; cellulose and its derivatives such as sodium carboxymethyl cellulose; ethyl cellulose and cellulose acetate; malt, gelatin, talcum, as well as other non-toxic compatible lubricants such as sodium lauryl sulfate and magnesium stearate; stabilizers, as well as coloring agents, solubilizers, adsorbents, antifoaming agents such as simethicone; releasing agents, viscosity builders such as carbomer-940; cetostearyl alcohol; flavoring and perfuming agents, surfactants, emulsifiers, anticaking agents, glidants, preservatives and antioxidants can also be present in the composition, according to the judgment of the formulator.

In formulating the ultimate composition, a number of conventional ingredients may be used. For instance, water; water miscible cosolvents, lanolin, Vaseline, glycerol, triglycerides of fatty acids, polyethylene glycols, oxyethyleneated fatty alcohols, metal oxides, saturated ketones such as camphor, alcohols such as menthol and phenoxy ethanol; esters such as isopropyl palmitate, myristate and stearate, sodium methyl paraben, sodium propyl paraben; ethers such as cetomacragol-1000; silicone oils, oleyl oleate, sorbitan mono oleate and butyl stearate; chelating agents such as disodium salt of EDTA; animal, vegetable, or mineral oils, fatty acids, glycerol monostearate, gels, amines such as triethanol amine; organic and mineral waxes. These ingredients are generally used in an amount of about 80-98% by weight of the total formulation and can be either a single or a multiple phase system.

Actual dosage levels of the active ingredients in the herbal compositions of this invention may be varied so as to obtain an amount of the bioactive ingredient, which is effective to achieve the desired therapeutic response for a particular patient, composition, and mode of administration without being toxic to the patient.

The selected dosage level will depend upon a variety of factors such as the duration of the treatment, combinations with the other bioactive substances, the age, sex, weight, condition, general health and prior medical history of the patient being treated, and like factors well known in the medical arts.

In another embodiment, the topical composition further comprises other plant extract exhibiting anti-dermatophyte activity in addition to the Murraya koenigii extract to obtain the synergistic effect. Plant may be selected from plants such as Curcuma zedoaria, Kaempferia galanga, Angelicae pubescentis Radix, Notopterygium Radix and Haliotis diversicolor Reeve.

In yet another embodiment, the topical composition further comprises other bioactive substances such as synthetic compound exhibiting anti-dermatophyte activity in addition to the Murraya koenigii extract to obtain a synergistic effect. Synthetic compounds may be selected from the anti-fungals, such as griseofulvin, tolnaftate, terbinafine hydrochloride and imidazoles such as miconazole nitrate and clotrimazole. The compositions of the present invention are suitable for use in the treatment of both acute and chronic forms of tinea infections, in particular the infections caused by Trichophyton, Epidermophyton and Microsporum species, in healthy and immunocompromised humans.

Unless defined otherwise, all technical and scientific terms used herein have the same meaning as commonly understood by one of ordinary skill in the art to which the invention belongs.

As used herein, “effective amount” means an amount of compound or composition (e.g., the Murraya koenigii extract) sufficient to significantly induce a positive modification in the condition to be regulated or treated within the scope of sound medical judgment. The effective amount of the compound or composition will vary with the particular condition being treated, the age and physical condition of the end user, the severity of the condition being treated/prevented, the duration of the treatment, the nature of concurrent therapy, the specific compound or composition employed, the particular cosmetically-acceptable carrier utilized, and like factors.

As used herein, all percentages are by weight unless otherwise specified.

The plant used in this study was collected from Karjat (Thane District) of Maharashtra, India. A microscopic and macroscopic study for authentication of Murraya koenigii was carried out. A specimen is retained in Botany department, Nicholas Piramal Research Centre, Goregaon, Mumbai, Maharashtra, India. The plant material was shade dried and coarse pulverized to 16-20 mesh size. The plant material was then extracted with organic solvents. The bioactivity guided purification and isolation of active ingredient was achieved as described in detail in subsequent examples.

The efficacy of the present plant extracts, compounds isolated by purification of the said extract, and formulations, was established by biological assays well known in the art and are described in detail in subsequent examples. The minimum inhibitory concentration (MIC) values were also determined.

The following examples illustrate but do not limit the scope of the invention. It is to be understood by those of the ordinary skill in the art that the present discussion is of exemplary embodiments only, and is not intended as limiting the broader aspects of the present invention, which broader aspects are embodied in the exemplary construction.

### EXAMPLE 1

### Preparation of Methanol Extract of Murraya Koenigii Roots

Shade dried roots (450 g) of Murraya koenigii were pulverized. The powdered material was extracted using methanol (4.5 L) at 60° C. for 3 hours. The extract was filtered under vacuum. This extraction process was repeated two more times. The extracts were combined and concentrated to remove the solvent.

Yield: 45.9 g (10.2% w/w)

The extract of example 1 was found to contain 7.74% of compound 1 (described in example 4), as estimated by HPTLC (solvent system:hexane:isopropyl alcohol (90:10); Quantification done at UV 204 nm).

### EXAMPLE 2

### Bioactivity by Agar Well Diffusion Method

The given extracts were tested against a panel of microorganisms, including Candida albicans 10231, Microsporum gypseum and Trichophyton mentagrophytes.

The strains were cultured overnight at 30° C. in Sabouraud dextrose agar. Colonies from subcultures were suspended in saline and vortexed to give a transmittance of 75-80% at 530 nm (T₅₃₀ nm≈10⁸ cfu/ml). Predecided volume of inoculum was mixed with molten agar at 40° C. and poured into sterile petri plates. Plates were allowed to set and solidify. Then wells (6 mm in diameter) were cut from the agar and 0.05 ml of extract solution was delivered into them. After incubation for 48 hours, at 28° C., all plates were examined for any zones of growth inhibition, and the diameters of these zones were measured in millimeters.

### Sample Preparation:

The stock solutions of 10 mg/ml of extracts of example 1 was prepared by suspending extract in methanol and sonicating for 10 seconds. Further dilutions were made to get a concentration range of 5, 2 and 1 mg/ml.

The results are summarized in table 1.

TABLE 1
Bioactivity of extracts

| | | | Zone of inhibiton in mm | | |
|---|---|---|---|---|---|
| Sr. | | Conc. | Trichophyton | Microsporum | Candida |
| No. | Sample | (mg/ml) | mentagrophytes | gypseum | ablicans |
| 1 | Extract of | 10 | 20 | 22 | 12 |
| | example 1 | 5 | 16 | 16 | 9 |
| | | 2 | 12 | 13 | NIL |
| | | 1 | 10 | 10 | NIL |

NIL: indicates no zone of inhibition
Conclusion: Extract of example 1 was found to be active against Trichophyton mentagrophytes and Microsporum gypseum.

### EXAMPLE 3

### Identification of the Bioactive Ingredient from Extract of Example 1

Identification of the bioactive ingredient from extract of example 1 was carried out by fractionation on preparative TLC as follows.

The extract of example 1 (20 mg) was loaded on silica gel preparative plate,

60 F 254, of thickness 2 mm. Hexane:ethyl acetate (75:25) was used as mobile phase. The bands were marked using UV 254 nm and 366 nm for detection. The bands were scraped out from the TLC plate and back extracted with methanol. The solvent was removed using high vacuum, and three fractions, fraction A, fraction B and fraction C were obtained.

Preparative TLC samples were analyzed for the activity by Agar well diffusion method as described in example 2.

The results are summarized as in table 2.

TABLE 2
Fractionation by preparative TLC and activity of fractions.

| Sr. | | Rf value | Trichophyton mentagrophytes |
|---|---|---|---|
| No. | Fraction | in TLC | (zone of inhibition in mm) |
| 1 | A | 0.703 | NIL |
| 2 | B | 0.644 | 22 |
| 3 | C | 0.381 | NIL |
| 4 | Extract of | | 20 |
| | Example 1 | | |

NIL: no zone of inhibition
Conclusion: The fraction B with Rf value 0.644 was a bioactive ingredient in extract of example 1.

### EXAMPLE 4

### Isolation of 2-methoxy-3-methyl-9H-carbazole (Compound 1) from Extract of Example 1

### Step 1

The extract of example 1 (46 g) was dissolved in 250 ml of petroleum ether with ultrasonication. The petroleum ether soluble fraction (yield: 26% w/w) and residue were subjected to TLC using hexane:ethyl acetate (75:25) as mobile phase. Based on the TLC profile, the petroleum ether soluble fraction showed the presence of compound corresponding to fraction B (Rf: 0.644) (As described in Example 3). The petroleum ether soluble fraction was separated, solvent was removed and semi-purified extract so obtained was used for step 2.

### Step 2

The semi-purified extract (1.5 g), prepared by the method described in step 1, was purified by column chromatography (silica gel, ethyl acetate in petroleum ether). The column yielded 380 mg of semi-pure material enriched with fraction B (Rf: 0.644).

### Step 3

Final purification of 380 mg semi-pure fraction (prepared as in step 2) was achieved by preparative HPLC.

Column: RP-18 (Eurosphere, 10%, 250×16 mm)

Solvent system: Gradient of acetonitrile:water

| Time | | |
|---|---|---|
| (min) | Acetonitrile % | Water % |
| 0 | 50 | 50 |
| 25 | 100 | 0 |
| 30 | 100 | 0 |
| 35 | 50 | 50 |
| 40 | 50 | 50 |

Flow rate: 15 ml/min.

UV detection: 210 nm.

Major peaks were obtained at retention time of 8.87, 13.42 and 21.27 min.

The peaks were collected and concentrated to dryness using vacuum.

All preparative HPLC fractions were analyzed for activity by Agar well diffusion method as described in example 2.

The fraction with retention time 8.87 was identified as the active fraction with zone of inhibition >25 mm (tested against Trichophyton mentagrophytes culture). Yield: 250 mg.

The active compound was characterized by comparing the obtained spectral data with the data given in the literature (Indian Journal of Chemistry, Vol. 24B, 452, (1985)).

The compound is identified as 2-methoxy-3-methyl-9H-carbazole. (Compound 1).

On analytical HPLC, fraction with retention time 8.87 in preparative HPLC and fraction B with Rf 0.644 on preparative TLC (as described in example 3), both had retention time of 14.28 min.

Analytical HPLC conditions:

Column: RP-18 (Eurosphere, 5μ, 125 mm×4.6 mm)

Solvent system: Gradient of acetonitrile:water

| Time | | |
|---|---|---|
| (min) | Acetonitrile % | Water % |
| 0 | 50 | 50 |
| 25 | 100 | 0 |
| 30 | 100 | 0 |
| 35 | 50 | 50 |
| 40 | 50 | 50 |

Flow rate: 1 ml/min

UV detection: 210 nm.

The data for 2-methoxy-3-methyl-9H-carbazole (Compound 1):

Nature: Yellowish oil

Yield: 250 mg

UV nm: 235, 255, 300 and 328;

IR cm⁻¹: 748 and 828 (substituted benzene derivative), 1038, 1230 (aromatic ether), 1305, 1504, 1612 and 1632 (aromatic residue) and 3419 (—NH);

MS: m/e (ES) 211 (M+).

¹H NMR (500 MHz, CDCl₃): δ, 2.56 (s, 3H), 4.02 (s, 3H), 6.76 (s, 1H), 7.22 (m, 1H), 7.28 (s, 1H), 7.45 (m, 1H), 7.50 (s, 1H), 8.00 (d, 1H) and 8.18 (s, 1H);

¹³C NMR (125 MHz, CDCl₃): δ, 22.13, 55.57, 107.9, 111.18, 112.77, 119.35, 120.64, 123.72, 124.55, 125.71, 128.22, 129.62, 139.71 and 145.52;

### EXAMPLE 5

### In Vitro Susceptibility Testing (Minimum Inhibitory Concentration) by Macro Broth Dilution Method—(CLSI Method-M-38).

In vitro susceptibility testing (Minimum Inhibitory Concentration) by macro broth dilution method—(CLSI method-M-38) was carried out as described in literature.

Reference method for broth dilution antifungal susceptibility testing of yeasts-Approved standard. NCCLS document M27-A. National Committee for Clinical Laboratory Standards, Wayne, Pa.;

Reference method for broth dilution antifungal susceptibility testing of filamentous fungi-Approved standard. NCCLS document M38-A. National Committee for Clinical Laboratory Standards, Wayne, Pa.

### Organisms:

Candida albicans, Trichophyton mentagrophytes and Micropsporum gypseum were used as test organisms.

### Medium:

RPMI 1640 (Sigma) with L-glutamine but without sodium bicarbonate and buffered at pH 7.0 with 3-(N-morpholino) propanesulfonic acid monosodium salt (MOPS), was the medium used for broth macro dilution susceptibility testing.

### Samples:

The extract of example 1, compound 1 and ketoconazole as standard.

### Sample Dilution:

The samples were prepared in methanol at 100 times the final concentration, followed by further dilutions (1:10) in RPMI 1640 medium to yield ten times the final strength required for the test. Purified component was also diluted in the same manner in methanol alone. Aliquots (0.1 ml) of each extract were dispensed into tubes. The final concentrations of extract ranged from 100-10 μg/ml.

### Inoculum Preparation:

Candida albicans:

A loopful of culture from 24-hour-old slant (37° C.) was suspended in saline. & vortexed for 15 seconds. The cell density of this suspension was adjusted by adding more saline & comparing it with transmittance of 0.5 McFarland standard at 530 nm. This yields a stock suspension of 1×10⁶ to 5×10⁶ cells/ml. A working suspension was made by a 1:100 dilution followed by a 1:20 dilution of the stock with RPMI medium, which results in 5×10² to 2.5×10³ cells/ml.

Trichophyton mentagrophytes and Micropsporum gypseum:

The cultures were grown on PDA agar slants at 28° C. for 7 days. The growth was covered with sterile saline and scraped to get a growth suspension. The resulting mixture of conidia or sporangiospores and hypal fragments was transferred to a sterile test tube. The heavy particles were allowed to settle and upper homogenous suspension was transferred to another tube. These suspensions were adjusted to an optical density that ranged from 0.09 to 0.11 (80 to 82% transmittance). These stock suspensions were further diluted 1:100 with medium to obtain test inoculum (0.4×10⁴ to 5×10⁴ cfu/ml).

### Broth Macro Dilution Testing:

Finally the tubes were inoculated with 0.1 ml of sample dilutions and 0.9 ml of the above-prepared inoculum. This results in 1:10 dilution of the sample and 10% dilution of the inoculum. Growth control tube received only diluent without sample. Positive control was set up using ketoconazole. A set of tubes of solvent control was also used.

### Incubation Time and Temperature:

The tubes were incubated at 37° C. for 24 hours for Candida albicans and 28° C. for 48 hours for other cultures.

### Endpoint Criteria:

The amount of growth in the tubes containing the sample was compared visually with the amount of growth in the growth control tubes. The minimum inhibitory concentration (MIC) was defined as the lowest concentration showing 100% growth inhibition.

The results are summarized in table 3.

TABLE 3
MIC values for extract of example 1 and compound 1

| | MIC values (μg/ml) | | |
|---|---|---|---|
| | Extract of | | |
| Organism | example 1 | Compound 1 | Ketoconazole |
| Trichophyton | 40 | 6 | 4 |
| mentagrophytes | | | |
| Microsporum | 40 | 4 | 4 |
| gypseum | | | |
| Candida | 80 | 30 | 5 |
| ablicans | | | |

Conclusion: MIC of compound 1 against dermatophytes is comparable with ketoconazole.

### Formulations

### EXAMPLE 6

### Preparation of Dusting Powder Formulation

The ingredients talcum, zinc oxide, aerosil, menthol, sodium benzoate, starch and perfume were weighed and mixed. Mixture was passed through sieve. (mesh size #40). Extract of example 1 was weighed and was added to the blend. The mixture was again passed through the sieve (mesh size #40) and was then packed in a suitable container.

TABLE 4
Each 50 g dusting powder contains

| Sr. No | Ingredients | Per batch (g) | % w/w |
|---|---|---|---|
| 01 | Extract of example 1 | 2.5 | 5 |
| 02 | Talcum | 39 | 78 |
| 03 | Zinc oxide | 2.5 | 5 |
| 04 | Aerosil | 1.25 | 2.5 |
| 05 | Menthol | 1.25 | 2.5 |
| 06 | Sodium benzoate | 0.25 | 0.5 |
| 07 | Starch | 2.5 | 5 |
| 08 | Perfume | 0.75 | 1.5 |

### EXAMPLE 7

### Preparation of Cream Formulation

### Step 1

### Preparation of Oil Phase

The ingredients ceostearyl alcohol, cetomacragol—1000, sorbitan mono oleate, self emulsifying glycerol monostearate, isopropyl myristate and stearic acid were weighed and were transferred into a suitable jacketed vessel. To it, weighed amount of extract of example 1 was added. The contents were melted at 70° C. and were mixed well.

### Step 2

### Preparation of Aqueous Phase

Demineralised water and carbomer-940 were mixed and to this gel triethanolamine was added and mass was mixed well. To it, di sodium EDTA, sodium lauryl sulphate and simethicone were added and mixed well. To this, sodium methyl paraben, sodium propyl paraben and phenoxy ethanol were added. This on mixing well gives aqueous phase.

### Step 3

The oil phase and aqueous phase were mixed and to it, propylene glycol, lilac jubo, camphor and methanol were added. The mass was cooled and filled in suitable container.

TABLE 5
Each 100 g cream contains

| Sr. | | Per batch | |
|---|---|---|---|
| No | Ingredients | (g) | % w/w |
| 01 | Extract of example 1 | 5.00 | 5.00 |
| 02 | Ceostearyl alcohol | 12.00 | 12.00 |
| 03 | Cetomacragol - 1000 | 3.00 | 3.00 |
| 04 | Sorbitan mono oleate | 2.00 | 2.00 |
| 05 | Self emulsifing glycerol monostearate | 3.00 | 3.00 |
| 06 | Isopropyl myristate | 2.50 | 2.50 |
| 07 | Stearic acid | 2.50 | 2.50 |
| 08 | Sodium methyl paraben | 0.40 | 0.40 |
| 09 | Sodium propyl paraben | 0.08 | 0.08 |
| 10 | Phenoxy ethanol | 0.52 | 0.52 |
| 11 | Di sodium EDTA | 0.02 | 0.02 |
| 12 | Carbomer-940 | 0.75 | 0.75 |
| 13 | Sodium lauryl sulphate | 0.75 | 0.75 |
| 14 | Simethicone | 0.75 | 0.75 |
| 15 | Triethanolamine | 1.00 | 1.00 |
| 16 | Propylene glycol | 5.00 | 5.00 |
| 17 | Lilac Jubo | 0.10 | 0.10 |
| 18 | Camphor | 0.50 | 0.50 |
| 19 | Methanol | 2.50 | 2.50 |
| 20 | Demineralised water | ~60.0* | ~60.0 |

*additional quantity of water is added to compensate for the loss due to evaporation during the process (if required)

### EXAMPLE 8

### Anti-Dermatophyte Activity for Murraya koenigii Formulations

MIC determination for dusting powder formulation (Example 6) and for cream formulation (example 7) was determined by in vitro susceptibility testing (Agar dilution method) as described in (Antimicrobial Agents And Chemotherapy; Vol. 41, No. 6, 1349-1351 (1997)).

The dusting powder mycoderm (FDC chemicals) was used as standard for dusting powder formulation.

Nizoral cream (Janssen-Cilag) was used as standard for cream formulation Organisms:

Trichophyton mentagrophytes and Micropsporum gypseum were used as test organisms.

### Inoculum Preparation:

Trichophyton mentagrophytes and Micropsporum gypseum:

The cultures were grown on PDA agar slants at 28° C. for 7 days. The growth was covered with sterile saline and scraped to get a growth suspension. The resulting mixture of conidia or sporangiospores and hypal fragments was transferred to a sterile test tube. The heavy particles were allowed to settle and upper homogenous suspension was transferred to another tube. These suspensions were adjusted to an optical density that ranged from 0.09 to 0.11 (80 to 82% transmittance) using a spectrophotometer. These stock suspensions were further diluted 1:10 with saline to obtain test inoculum (1×10³ to 5×10³ cfu/ml).

### Sample Preparation:

A stock solution of formulation was prepared in hexane. Calculated amount of stock solution was added to 15 ml of Sabarouds melted agar medium and poured into petri plates so as to get a series of serial two fold dilution of the extract in the medium. The final concentrations of formulation in the plate medium ranged from 1 to 0.05 mg/ml. A solvent control plate was also included. A growth control plate without any extract or formulation was included in the study.

### Assay:

The culture suspensions prepared by above method were spotted in 101 amount on the solidified plates. Spots were allowed to dry at room temperature and then the plates were incubated at 28° C. for 48 hours.

### End Point Criteria:

The MIC was defined as the lowest concentration of extract or formulation giving no visible growth or causing almost complete inhibition of growth in the plates.

The results are summarized in table 6 and table 7

TABLE 6
MIC (mg/ml) values for dusting powder formulation (example 6)

MIC values (mg/ml)

| | | Dusting powder |
|---|---|---|
| Organism | Example 6 | Mycoderm |
| Trichophyton | 0.3 | 0.4 |
| mentagrophytes | | |
| Microsporum | 0.2 | 0.3 |
| gypseum | | |

TABLE 7
MIC (mg/ml) values for cream formulation (example 7)

MIC values (mg/ml)

| Organism | Example 7 | Nizoral cream |
|---|---|---|
| Trichophyton | 0.4 | 0.5 |
| mentagrophytes | | |
| Microsporum | 0.4 | 0.5 |
| gypseum | | |

1. A herbal composition comprising an effective amount of an extract obtained from the roots of the plant Murraya koenigii, and a pharmaceutically acceptable carrier, for the treatment of an infection caused by dermatophytes.

2. The herbal composition as claimed in claim 1, wherein the infection is tinea infection.

3.-4. (canceled)

5. The herbal composition as claimed in claim 1, wherein said extract of Murraya koenigii is provided in an amount of 2.0 to 20% by weight.

6. The herbal composition as claimed in claim 5, wherein the extract of Murraya koenigii is provided in an amount of 2.5 to 10% by weight.

7. The herbal composition as claimed in claim 1, wherein the said extract of Murraya koenigii contains an effective amount of a bioactive ingredient.

8. The herbal composition as claimed in claim 7, wherein said bioactive ingredient is present in 0.1-2.0% by weight.

9.-10. (canceled)

11. The herbal composition as claimed in claim 7, wherein the said bioactive ingredient is 2-methoxy-3-methyl-9H-carbazole.

12. (canceled)

13. The herbal composition as claimed in claim 2, wherein the tinea infection is caused by Trichophyton mentagrophytes or Microsporum gypseum.

14. (canceled)

15. The herbal composition as claimed in claim 1, wherein the composition is formulated for topical administration.

16. The herbal composition as claimed in claim 15, wherein the composition for topical administration is in the form of dusting powder.

17. The herbal composition as claimed in claim 15, wherein the composition for topical administration is in the form of a cream.

18. A method for the treatment of an infections caused by dermatophytes comprising administering to a person in need thereof an effective amount of an herbal composition comprising:
An effective amount of an extract obtained from the roots of the plant Murraya koenigii, and a pharmaceutically acceptable carrier, for the treatment of an infection caused by dermatophytes.

19. The method of claim 18, wherein the infection is tinea infection.

20. (canceled)

21. The method of claim 19, wherein the tinea infection is caused by Trichophyton mentagrophytes or Microsporum gypseum.

22. The method of claim 18, wherein the said composition is formulated for topical administration.

23. An herbal composition as claimed in claim 1, wherein said composition further comprises an herbal extract exhibiting anti-dermatophyte activity.

24. An herbal composition as claimed in claim 1, wherein said composition further comprises a bioactive substance exhibiting anti-dermatophyte activity.
